(19) United States
(12) Patent Application Publication
(10) Pub. No.: US 2007/0259861 A1
(43) Pub. Date: Nov. 8, 2007

(54) Combination therapy with non-selective COX inhibitors to prevent COX-related gastric injuries

(75) Inventor: Alexander Krantz, Boston, MA (US)

Correspondence Address: DRINKER BIDDLE & REATH LLP;One Logan Square, 18th & Cherry Streets, Philadelphia, PA, 19103-6996, US

(73) Assignee: Pharmena North America Inc., Ottawa (CA)

(21) Appl. No.: 11/715,660

(22) Filed: Mar. 8, 2007

Related U.S. Application Data
(60) Provisional application No. 60/780,264, filed on Mar. 8, 2006.

(51) Int. Cl. A61K 31/4439; A61K 31/19; A61K 31/192; A61K 31/196; A61P 29/00; A61K 31/5415; A61K 31/235; A61K 31/40

(52) U.S. Cl. 514/226.5; 514/340; 514/413; 514/548; 514/567; 514/568; 514/569

(57) ABSTRACT

The present invention is directed to nicotinamide, nicotinamide derivatives and prostaglandin mimetics, alone or in combination with an NSAID, and their use in treating pain, inflammation, and/or gastrointestinal toxicty,

### RELATED APPLICATION

This application claims priority to U.S. Provisional Application No. 60/780,264, Attorney Docket No. PRI-004-1, filed Mar. 8, 2006, titled “COMBINATION THERAPY WITH NON-SELECTIVE COX INHIBITORS TO PREVENT COX-RELATED GASTRIC INJURIES,” which is incorporated herein by reference in its entirety. Additionally, the contents of any patents, patent applications, and references cited throughout this specification are hereby incorporated by reference in their entireties.

### BACKGROUND OF THE INVENTION

Non-steroidal anti-inflammatory drugs (NSAIDs), including compounds such as ibuprofen, ketoprofen and diclofenac, have anti-inflammatory actions and are effective on pain associated with the release of prostaglandins and other mediators of inflammation. For example, diclofenac is considered to be extremely potent and effective as an analgesic and anti-inflammatory agent. Diclofenac is approved in the United States for the long-term symptomatic treatment of rheumatoid arthritis, osteoarthritis and alkylosing spondylitis. It is also considered to be useful for the short-term treatment of acute musculoskeletal injury, acute painful shoulder, postoperative pain and dysmenorrhea. Furthermore, NSAIDs have been widely used in arthritis therapy for several years.

Although NSAIDs are widely accepted as effective agents for controlling pain, their administration can lead to the development of gastroduodenal lesions, e.g., ulcers and erosions, in susceptible individuals. It has been suggested that a major factor contributing to the development of these lesions is the presence of acid in the stomach and upper small intestine of patients. This view is supported by clinical studies demonstrating an improvement in NSAID tolerability when patients are also taking independent doses of acid inhibitors (Dig. Dis. 12:210-222 (1994); Drug Safety 21:503-512 (1999); Aliment. Pharmacol. Ther. 12:135-140 (1998); Am. J. Med. 104(3A):67S-74S (1998); Clin. Ther. 17:1159-1173 (1995)). Other major factors contributing to NSAID-associated gastropathy include a local toxic effect of NSAIDs and inhibition of protective prostaglandins (Can. J. Gastroenterol. 13:135-142 (1999) and Pract. Drug Safety 21:503-512, (1999)), which may also make some patients more susceptible to the ulcerogenic effects of other noxious stimuli.

Attempts to develop NSAIDs that are inherently less toxic to the gastrointestinal tract have met with only limited success. For example, the recently developed cyclooxygenase-2 (COX-2) inhibitors show a reduced tendency to produce gastrointestinal ulcers and erosions, but a significant risk is still present, especially if the patient is exposed to other ulcerogens (JAMA 284:1247-1255 (2000); N. Eng. J. Med. 343:1520-1528 (2000)). In addition, the COX-2 inhibitors may not be as effective as other NSAIDs at relieving some types of pain and have been associated with significant cardiovascular problems (JADA 131:1729-1737 (2000); SCRIP 2617, pg. 19, Feb. 14, 2001).

Other attempts to produce an NSAID therapy with less gastrointestinal toxicity have involved the concomitant administration of a cytoprotective agent. In 1998, Searle began marketing Arthrotec™ for the treatment of arthritis in patients at risk for developing GI ulcers. This product contains misoprostol (a cytoprotective prostaglandin) and the NSAID diclofenac. Although patients administered Arthrotec™ do have a lower risk of developing ulcers, they may experience a number of other serious side effects such as diarrhea, severe cramping and, in the case of pregnant women, potential damage to the fetus.

Another approach has been to produce enteric coated NSAID products. However, even though these have shown modest reductions in gastroduodenal damage in short term studies (Scand. J. Gastroenterol. 20: 239-242 (1985) and Scand. J. Gastroenterol. 25:231-234 (1990)), there is no consistent evidence of a long term benefit during chronic treatment.

Overall, it may be concluded that the risk of gastrointestinal toxicity in the form of gastritis, peptic erosions, ulcerations, GI bleeds, etc., is a recognized problem associated with the administration of NSAIDs and that, despite considerable effort, an ideal solution has not yet been found.

### SUMMARY OF THE INVENTION

In one aspect, the invention provides a pharmaceutical composition comprising an NSAID and a prostaglandin mimetic. In one embodiment, the prostaglandin mimetic is selected from the group consisting of genistein, daidzein, tamoxifen, tetrandrine, thapsigargin and a compound of formula (I): 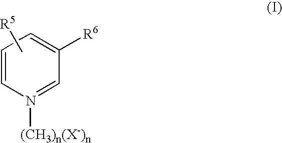
wherein n is 0 or 1; R⁵ is H, F or Cl; R⁶ is azido, —C(O)CHN₂, —N(H)C(O)NH₂, —N(H)C(O)H, —C(O)R or the following 5-membered heterocycle: 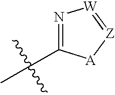
wherein R is NR²R³ or OR⁴; R² and R⁴ each, independently, are hydrogen or C₁₋₄alkyl; R³ is hydrogen, C₁₋₄alkyl or CH₂OH; W and Z are, independently, C(H) or N; A is O, S or NH; and X⁻ is a physiologically suitable counter-anion.”

In another embodiment, the prostaglandin mimetic is a prostacyclin mimetic. In yet another embodiment, the prostaglandin mimetic is a prostaglandin agonist. In still another embodiment, prostaglandin agonist is selected from a PGI₂ agonist or a PGE₂ agonist. In another embodiment, the agonist is selected from the group consisting of U46619, I-BOP, STA₂, BW245C, L-644698, ZK110841, 13,15-dihydro-15-keto-PGD₂, indomethacin, 15-R-methyl-PGD₂, 15d-PGJ₂, ONO-KI-004, iloprost, 17-phenyl-trinor PGE₂, sulprostone, butaprost, 11-deoxy PGE₁, AH13205, ONO-AEI-259, sulprostone, MB28767, misoprostol, SC46275, ONO-AE-249, PGE₁-OH, ONO-AEI-329, cicaprost, carbacyclin, fluprostenol, latanoprost, travoprost, bimatoprost, beraprost, cloprostenol sodium, eicosopentanoic acid, docosohexanoic acid, ceramide, sodium butyrate and aluminum fluoride.

In another embodiment, the invention provides use of the pharmaceutical composition of the invention, for the treatment or prevention of deleterious effects associated with NSAID administration in a subject. In one embodiment, the deleterious effects associated with NSAID administration in a subject is related to a decrease in one or more prostaglandins anywhere in the gastrointestinal tract. In another embodiment, the deleterious effects associated with NSAID administration in a subject is related to a decrease in one or more prostaglandins in the stomach. In still another embodiment, the prostaglandins are PGE₂ and/or PGI₂. In another embodiment, the deleterious effect associated with NSAID administration in a subject is GI toxicity. In yet another embodiment, the GI toxicity is selected from the group consisting of gastritis, peptic erosions, ulceration, gastric lesions and GI bleeds.

In another embodiment, the NSAID for use in the invention is selected from the group consisting of aspirin, indomethacin, voltaren, naprosyn, ibuprofen, naproxen, fenoprofen, tolmetin, sulindac, meclofenamate, ketoprofen, piroxicam, flurbiprofen, diclofenac, acetylsalicylic acid, sodium acetylsalicylic acid, calcium acetylsalicylic acid, salicylic acid, sodium salicylate, choline salicylate, magnesium salicylate, salsalate, sodium salicylate, diflunisal, ketorolac, carprofen, mefenamic acid, meloxicam and nimesulide.

In another aspect, the invention provides a method of treating pain and/or inflammation in a subject, said method comprising administering to the subject in need thereof a therapeutically-effective amount of a pharmaceutical composition comprising an NSAID and a molecule that selectively stimulates the release of a prostaglandin. In one embodiment, the molecule is selected from the group consisting of genistein, daidzein, tamoxifen, tetrandrine, thapsigargin and a compound of formula (I).

In another aspect, the invention provides a method of treating pain and/or inflammation in a subject, said method comprising administering to the subject in need thereof a therapeutically-effective amount of a pharmaceutical composition comprising an NSAID and a prostaglandin agonist. In one embodiment, the prostaglandin agonist is selected from a PGI₂ agonist or a PGE₂ agonist. In another embodiment, the agonist is selected from the group consisting of U46619, I-BOP, STA₂, BW245C, L-644698, ZK110841, 13,15-dihydro-15-keto-PGD₂, indomethacin, 15-R-methyl-PGD₂, 15d-PGJ₂, ONO-KI-004, iloprost, 17-phenyl-trinor PGE₂, sulprostone, butaprost, 11-deoxy PGE₁, AH13205, ONO-AEI-259, sulprostone, MB28767, misoprostol, SC46275, ONO-AE-249, PGE₁-OH, ONO-AEI-329, cicaprost, carbacyclin, fluprostenol, latanoprost, travoprost, bimatoprost, beraprost, cloprostenol sodium, eicosopentanoic acid, docosohexanoic acid, ceramide, sodium butyrate and aluminum fluoride. In another embodiment, the inflammation is selected from the group consisting of fever, arthritis, asthma, bronchitis, menstrual cramps, tendinitis, bursitis, inflammatory disorders of the skin, gastrointestinal conditions, vascular diseases, migraine headaches, periarteritis nodosa, thyroidiris, aplastic anemia, Hodgkin's disease, sclerodoma, rheumatic fever, myasthenia gravis, sarcoidosis, nephrotic syndrome, Behcet's syndrome, polymyositis, hypersensitivity, conjunctivitis, gingivitis, swelling occurring after injury and myocardial ischemia. In yet another embodiment, the arthritis is selected from rheumatoid arthritis, spondyloarthopathies, gouty arthritis, systemic lupus erythematosus, osteoarthritis and juvenile arthritis. In still another embodiment, the inflammatory disorders of the skin are selected from the group consisting of psoriasis, eczema, burns and dermatitis. In another embodiment, the gastrointestinal conditions are selected from the group consisting of inflammatory bowel syndrome, Crohn's disease, gastritis, irritable bowel syndrome and ulcerative colitis. In yet another embodiment, the pain is selected from the group consisting of menstrual pain, low back pain, neck pain, skeletal pain, post-partum pain, headache, pain associated with migraine, toothache, sprains, strains, arthritis, degenerative joint diseases, gout, ankylosing spondylitis, bursitis, burns, including radiation and corrosive chemical injuries, sunburns, bone fracture, immune and autoimmune diseases, cellular neoplastic transformations or metastic tumor growth, and pain following surgical and dental procedures. In another embodiment, the arthritis is selected from rheumatoid arthritis, spondyloarthopathies, gouty arthritis, systemic lupus erythematosus, osteoarthritis and juvenile arthritis. In still another embodiment, the therapeutically-effective amount of a pharmaceutical composition comprising an NSAID and a compound of formula (I) is used to treat gastric lesions, symptoms of GI toxicity, ischemia, reperfusion, colorectal cancer and damage to the gastric mucosa in a subject. In yet another embodiment, the symptoms of GI toxicity are selected from the group consisting of gastritis, peptic erosions, ulcerations and GI bleeding.

In another aspect, the invention provides a method of treating pain and/or inflammation in a subject, said method comprising administering to the subject in need thereof a therapeutically-effective amount of a compound of formula (I).

In one embodiment of the compound of formula (I), n is 1. In another embodiment R⁶ is —C(O)R, and R is NR²R³. In still another embodiment, R⁶ is —C(O)R, R is NR²R³, and R² represents methyl or hydrogen. In yet another embodiment, R⁶ is —C(O)R, R is NR²R³, and R³ represents CH₂OH or hydrogen. In another embodiment, R⁶ is —C(O)R, R represents the group OR⁴, and R⁴ represents C₁₋₄ alkyl. In yet another embodiment, R⁶ is —C(O)R, R represents the group OR⁴, and R⁴ represents propyl or ethyl. In still another embodiment, the compound of formula (I) is selected from a 1-methylnicotinamide salt or a 1-methyl-N′-hydroxymethylnicotinamide salt. In another embodiment, the compound of formula (I) is selected from a 1-methylnicotinic acid ethyl ester salt or a 1-methylnicotinic acid propyl ester salt. In yet another embodiment, the compound of formula (I) is selected from a 1-methylnicotinic acid salt. In still another embodiment, n is 1 and X⁻ is chloride, benzoate, salicylate, acetate, citrate or lactate. In another embodiment, the compound of formula (I) is selected from 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride or 1-methylnicotinic acid propyl ester chloride. In yet another embodiment, R⁶ is a 5-membered heterocycle, wherein A is O, and W and Z are CH; A is S, and W and Z are CH; A is NH, and Wand Z are CH; A is O, W is N and Z is CH; A is S, W is N and Z is CH; A is NH, W is N and Z is CH; A is O, W is CH and Z is N; A is S, W is CH and Z is N; A is NH, W is CH and Z is N; A is O, W and Z are CH; A is S, W and Z are CH; or A is NH and W and Z are N. Instill another embodiment, n is 1. In another embodiment, n is 1, R⁵ is H and R⁶ is azido.

In another aspect, the invention provides a method of treating gastric lesions, symptoms of GI toxicity, ischemia, reperfusion, colorectal cancer or damage to the gastric mucosa in a subject in need thereof by administering to the subject a pharmaceutical composition comprising an NSAID and a 1-methylnicotinamide salt. In a particular embodiment, the symptoms of GI toxicity are selected from the group consisting of gastritis, peptic erosions, ulcerations and GI bleeding.

In another aspect, the invention provides a method of treating an inflammation-associated disorder and/or pain in a subject in need thereof by administering to the subject a pharmaceutical composition comprising an NSAID and a 1-methylnicotinamide salt, and pharmaceutically acceptable salts thereof.

### DETAILED DESCRIPTION OF THE INVENTION

The present invention is directed to nicotimamide and nicotinamide derivatives. Furthermore, the present invention is directed toward a pharmaceutical composition comprising one or more NSAIDs and a prostaglandin mimetic, and its use in treating diseases and disorders, such as those related to pain and inflammation. In one embodiment, the prostaglandin mimetic selectively stimulates the release of PGI₂ or PGE₂. In a particular embodiment, the prostaglandin mimetic is nicotimamide or a nicotinamide derivative. In another embodiment, the prostaglandin mimetic is a prostaglandin (e.g., PGI₂ or PGE₂) agonist. In a further embodiment, the prostaglandin mimetic serves to counteract the deleterious side effects associated with NSAID administration. In particular, specific embodiments of the invention are described herein as exemplary embodiments and are not intended to be limiting.

Complications arising from chronic NSAID use are common and are primarily due to gastrointestinal (GI) toxicity in the form of, for example, gastritis, peptic erosions, ulceration, gastric lesions and GI bleeds. This GI toxicity has been attributed to the blockade of the isoform cyclo-oxygenase (COX)-1 which is the primary isoform responsible for the production of cytoprotective prostaglandins (PGE₂ and PGI₂) in the stomach. However, this hypothesis appears to be oversimplified.

In a series of articles dealing with selective and unselective COX inhibitors (reviewed in “COX inhibition and NSAID-induced gastric damage-roles in various pathogenic events.” Curr Top Med. Chem. 2005; 5(5):475-86) Takeuchi et al. have investigated the relation between COX inhibition and pathogenic events in rodents. Takeuchi et al. conclude that (1) the gastric ulcerogenic properties of NSAIDs are not accounted for solely by the inhibition of COX-1 and require the inhibition of both COX-1 and COX-2, (2) the inhibition of COX-1 up-regulates COX-2 expression in association with gastric hypermotility, and (3) prostaglandins produced by COX-2 counteract the deleterious influences of the COX-1 inhibition.

In light of these observations, a strategy designed to stimulate the production of one or more prostaglandins in a subject that are otherwise depleted by the administration of NSAIDs is warranted, and is the subject of this invention. Without being bound by theory, it is believed that the stimulation of these prostaglandins serves to counteract the contributions of NSAID-induced COX inhibition to injuries of the gastrointestinal tract. The production of such depleted prostaglandins can be amplified through the use of a prostaglandin mimetic. In one embodiment, the prostaglandin mimetic selectively stimulates the release of PGI₂ or PGE₂. In a particular embodiment, the prostaglandin mimetic is nicotimamide or a nicotinamide derivative of the Formula (I). In another embodiment, the prostaglandin mimetic is a prostaglandin (e.g., PGI₂ or PGE₂) agonist. In a certain embodiment, the prostaglandin mimetic is administered with one or more NSAIDs (e.g., naprosyn, voltaren, or indomethacin) to a subject as a pharmaceutical composition.

For example, which is not intended to be limiting, the major prostaglandins produced by COX-2 are PGE₂ and PGI₂. As discussed above, NSAID-induced depletion of either of these prostaglandins may lead to GI toxicity. Therefore, replenishing the concentrations of either or both of these prostaglandins will counteract these adverse effects. For example, N-methylnicotinamide (MNA, also referred to as 1-methylnicotinamide) has been demonstrated in a thrombolytic assay to stimulate the secretion of PGI₂ (WO05067927A3; incorporated herein by reference). Thus, MNA could be co-administered, in appropriate proportions, with one or more NSAIDs to counteract adverse effects in the GI tract by the enhanced production of PGI₂. A pharmaceutical composition of this sort will also be effective for the treatment of NSAID-associated disease and disorders such as pain and inflammation.

Similarly, a pharmaceutical composition comprising one or more NSAIDs and a prostaglandin mimetic can be used for the treatment of NSAID-associated diseases and disorders such as pain and inflammation.

Definitions

These and other embodiments of the invention will be described with reference to following definitions that, for convenience, are collected here.

As used herein, the term “NSAID” includes, but is not limited to, those agents which inhibit cyclooxygenase, the enzyme responsible for the biosyntheses of the prostaglandins and certain autocoid inhibitors, including inhibitors of the various isoenzymes of cyclooxygenase (including, but not limited to, cyclooxygenase-1 and -2), such as the commercially available NSAIDs aceclofenac, acemetacin, acetaminophen, acetaminosalol, acetyl-salicylic acid, acetyl-salicylic-2-amino-4-picoline-acid, 5-aminoacetylsalicylic acid, alclofenac, aminoprofen, amfenac, ampyrone, ampiroxicam, anileridine, bendazac, benoxaprofen, bermoprofen, α-bisabolol, bromfenac, 5-bromosalicylic acid acetate, bromosaligenin, bucloxic acid, butibufen, carprofen, celecoxib, chromoglycate, cinmetacin, clindanac, clopirac, sodium diclofenac, diflunisal, ditazol, droxicam, enfenamic acid, etodolac, etofenamate, felbinac, fenbufen, fenclozic acid, fendosal, fenoprofen, fentiazac, fepradinol, flufenac, flufenamic acid, flunixin, flunoxaprofen, flurbiprofen, glutametacin, glycol salicylate, ibufenac, ibuprofen, ibuproxam, indomethacin, indoprofen, isofezolac, isoxepac, isoxicam, ketoprofen, ketorolac, lomoxicam, loxoprofen, meclofenamic acid, mefenamic acid, meloxicam, mesalamine, metiazinic acid, mofezolac, montelukast, mycophenolic acid, nabumetone, naproxen, niflumic acid, nimesulide, olsalazine, oxaceprol, oxaprozin, oxyphenbutazone, paracetamol, parsalmide, perisoxal, phenyl-acethyl-salicylate, phenylbutazone, phenylsalicylate, pyrazolac, piroxicam, pirprofen, pranoprofen, protizinic acid, reserveratol, salacetamide, salicylamide, salicylamide-O-acetyl acid, salicylsulphuric acid, salicin, salicylamide, salsalate, sulindac, suprofen, suxibutazone, tamoxifen, tenoxicam, theophylline, tiaprofenic acid, tiaramide, ticlopridine, tinoridine, tolfenamic acid, tolmetin, tropesin, xenbucin, ximoprofen, zaltoprofen, zomepirac, tomoxiprol, zafirlukast and cyclosporine. Additional NSAID genera and particular NSAID compounds are disclosed in U.S. Pat. No. 6,297,260, incorporated entirely by reference (especially in the generic formulas of its claim 1 and the recitation of specific list of NSAIDs contained therein and in claim 3, and thiazulidene NSAIDs disclosed in International Patent Application WO 01/87890, incorporated herein by reference in its entirety). Preferred NSAIDs are indomethacin, flufenamic acid, flunixin and theophylline. Most preferred is indomethacin, voltaren and naprosyn. In certain embodiments, the NSAID subunit is neither acetyl salicylic acid or mycophenolic acid. Additionally, The Merck Manual, 16th Edition, Merck Research Laboratories (1990) pp 1308-1309, as well as The Pharmacological Basis of Therapeutics, 9th edition, Macmillan Publishing Co., 1996, pp 617-655, provide well known examples of NSAIDs.

As used herein, the term “prostaglandin mimetic” refers to a molecule such as a compound, a drug, an enzyme activator or a hormone that mimics a property of a prostaglandin or mimics a prostaglandin. Examples of prostaglandin mimetics include, but are not limited to, selective prostaglandin mimetics, e.g., nicotimamide, or nicotinamide derivatives of the invention such as MNA, and prostaglandin agonists. Without being bound by theory, it is believed that the stimulation of the release of particular prostaglandins counteract the deleterious effects (or a symptom of the deleterious effects) associated with NSAID use (e.g., GI toxicity).

The language “deleterious effects associated with NSAID use,” as used herein, describes diseases and disorders that may be treated or prevented (or a symptom of such disease or disorder that may be reduced) by the prostaglandin mimetics of the invention in that the prostaglandin mimetics mimic a prostaglandin in a subject, e.g., a property or mode of action of a prostaglandin in a subject. A prostaglandin mimetic could be a molecule that is directly responsible for the release of a prostaglandin, or elevation of a prostaglandin concentration in a subject, or the enhanced expression of a gene capable of elevating prostaglandins levels in a subject. A further example of a prostaglandin mimetic is a molecule that acts directly on a prostacyclin receptor, such as a prostaglandin agonist, such as a prostaglandin 12 (IP) receptor agonist.

Alternatively, the “deleterious effects associated with NSAID use” can describe diseases and disorders that may be treated or prevented (or a symptom of such disease or disorder that may be reduced) by the prostaglandin mimetics of the invention in that the prostaglandin mimetics selectively stimulate the release of a prostaglandin or lead to the enhanced expression of a gene capable of elevating prostaglandins levels. A prostaglandin mimetic that “selectively stimulates the release of a prostaglandin” does not act directly on receptors that bind prostaglandins but creates elevated levels of a particular prostaglandin that are available for the increased binding of that prostaglandin to its target receptor(s). Such prostaglandin mimetics are referred to herein as “selective prostaglandin mimetics.” Non-limiting examples of selective prostaglandin mimetics include the nicotinamide derivatives of the invention (e.g., nicotimamide, MNA), genistein, daidzein (see, e.g., Journal of Pharmacology and Experimental Therapeutics Fast Forward; DOI: 10.1124/jpet.105.090456), tamoxifen (see, e.g., BMC Cancer 2003, 3:24; DOI: 10.1186/1471-2407-3-24), tetrandrine, thapsigargin (see, e.g., BMC Pharmacol. 2005; 5: 12.; DOI: 10.1186/1471-2210-5-12), as well as the PGI₂ production promoters described in U.S. Pat. No. 5,530,001, all of which are incorporated herein by reference in their entirety.

In certain embodiments, the prostaglandin mimetic mimics a property or mode of action of prostacyclin.

In certain embodiments, the stimulation of the release of a prostaglandin by a prostaglandin mimetic of the invention may account for greater than 10%, e.g., greater than 20%, e.g., greater than 30%, e.g., greater than 40%, e.g., greater than 50%, of the treatment, prevention, or reduction of the symptoms of the deleterious effects associated with NSAID use. In certain embodiments, the stimulation of the release of a prostaglandin may result in one or more effects that account for said percentage of the treatment, prevention, or reduction of the symptoms of the deleterious effects associated with NSAID use. Similarly, the deleterious effects associated with NSAID use may be alleviated using the prostaglandin mimetics of the invention to replenish the amount of the depleted prostaglandin to pre-NSAID administration levels. In another embodiment, the depleted prostaglandin levels are brought to levels that are greater than pre-NSAID administration levels, e.g., greater than 100% of the pre-NSAID administration levels. In other embodiments, the depleted prostaglandin levels are brought to levels that are greater than 1%, e.g., greater than 5%, e.g., greater than 10%, e.g., greater than 20%, e.g., greater than 30%, e.g., greater than 40%, e.g., greater than 50%, e.g., greater than 60%, e.g., greater than 70%, e.g., greater than 80%, e.g., greater than 90% of the pre-NSAID administration levels.

The term “prostaglandin agonist” refers to a compound which binds to prostaglandin receptors (e.g., An S. et al., Cloning and Expression of the EP₂ Subtype of Human Receptors for Prostaglandin E₂, Biochemical and Biophysical Research Communications, 1993, 197(1):263-270) and mimics the action of at least one prostaglandin in vivo. A prostaglandin agonist also includes prostaglandin I₂(IP) receptor agonists. Examples of such IP receptor antagonists can be found in U.S. Pat. Nos. 6,998,414, and 6,335,459, which are incorporated herein by reference. Additionally, a prostaglandin agonist includes compounds that bind to the nuclear receptor peroxisome proliferator-activated receptor (PPAR; see, e.g., Am. J. Respir. Cell Mol. Biol., 34, (2), 2006, 242-246). PPAR agonists may include agonists of any of the PPAR subunits or combinations thereof. For example, the PPAR agonist may include agonists of PPARα, PPARγ, PPARδ, PPARα+γ, or PPARα+δ, for example a thiazolidinedione or a fibrate. Thiazolidinediones for use in combination with the prostaglandin mimetics of the invention include, but are not limited to, 5-((4-(2-(methyl-2-pyridinylamino)ethoxy)phenyl)methyl)-2,4-thiazolidinedione, troglitazone, pioglitazone, ciglitazone, WAY-120, 744, englitazone, AD 5075, darglitazone, and rosiglitazone. Fibrates for use in combination with the prostaglandin mimetics of the invention include, but are not limited to, gemfibrozil, fenofibrate, clofibrate, or ciprofibrate.

Prostaglandin agonist compounds include natural prostaglandins such as PGI₂, PGD₁, PGD₂, PGE₂, PGE₁, PGF₂ and PGF₂α, and analogs of the natural prostaglandins, including compounds that bind to PPAR, the PGI₂ receptor (IP), the PGE₂ receptors (EP₁, EP₂, EP₃ and EP₄), the TXA₂ receptor (TP), the PGD₂ receptors (DP and CRTH2) and the PGF_{2α} receptor (FP). An example of a prostacyclin analog that is useful for the purposes of this invention is U-68,215 (see, e.g., J Pharmacol Exp Ther. 1992 March; 260(3):1023-7). In a preferred embodiment, the prostaglandin agonist is a compound that bind to the PGI₂ receptor (IP) and/or the PGE₂ receptors (EP₁, EP₂, EP₃ and EP₄). Certain prostaglandin agonists are disclosed in GB 1478281, GB1479156 and U.S. Pat. Nos. 4,175,203, 4,055,596, 4,175,203, 3,987,091, 3,991,106, 6,498,172 and Pharmacol Ther. 2004 103, 147-66 (all of which are incorporated herein by reference). Examples of prostaglandin agonists useful for the purposes of this invention include, but are not limited to, U46619, I-BOP and STA₂ (agonists of TXA₂), BW245C, L-644698, ZK110841, 13,15-dihydro-15-keto-PGD₂, indomethacin, 15-R-methyl-PGD₂, 15d-PGJ₂ (agonists of PGD₂), ONO-KI-004, iloprost, 17-phenyl-trinor PGE₂, sulprostone, butaprost, 11-deoxy PGE₁, AH13205, ONO-AEI-259, sulprostone, MB28767, misoprostol, SC46275, ONO-AE-249, PGE₁-OH, and ONO-AEI-329 (PGE₂ agonists), iloprost, cicaprost and carbacyclin, (agonists of PGI₂), fluprostenol and latanoprost (agonists of PGF_{2α}), as well as travoprost, bimatoprost, beraprost, carbacyclin, cloprostenol sodium, eicosopentanoic acid, docosohexanoic acid, ceramide, sodium butyrate and aluminum fluoride. Preferred prostaglandin agonists of the invention include agonists of PGI₂ and PGE₂. Particularly preferred prostaglandin agonists of the invention are agonists of PGI₂.

“Agonist” refers to a molecule such as a compound, a drug, an enzyme activator or a hormone that enhances the activity of another molecule or receptor site.

“NSAID-associated diseases and disorders” refers to any disease, disorder or condition that may be treated in a subject in need thereof with NSAID administration. Examples of NSAID-associated diseases and disorders include, but are not limited to, pain and inflammation.

As used herein, the term “pain” includes all types of pain. Pain includes, but is not limited to, chronic pains, such as arthritis pain (e.g., pain associated with osteoarthritis and rheumatoid arthritis), neuropathic pain, and post-operative pain, chronic lower back pain, cluster headaches, herpes neuralgia, phantom limb pain, central pain, dental pain, neuropathic pain, opioid-resistant pain, visceral pain, surgical pain, bone injury pain, pain during labor and delivery, pain resulting from burns, including sunburn, post partum pain, migraine, angina pain, and genitourinary tract-related pain including cystitis. The term also refers to nociceptive pain or nociception. The term shall refer to persistent pains, such as, but not limited to, neuropathic pain, diabetic neuropathy, fibromyalgia, pain associated with somatoform disorders, arthritic pain, cancer pain, neck pain, shoulder pain, back pain, cluster headaches, tension-type headache, migraine, herpes neuralgia, phantom limb pain, central pain, dental pain, pain traditionally resistant to treatment with NSAIDs, and post-operative pain. The term pain also refers to the pain associated with the inflammation-related diseases and disorders described herein.

The term “inflammation” and “inflammation-related disease and disorder” will be understood by those skilled in the art to include any condition characterized by a localized protective response elicited by injury or destruction of tissues resulting from any of the causes mentioned hereinbefore, and which is manifest by heat, swelling, pain, redness, dilation of blood vessels and/or increased blood flow, invasion of the affected area by white blood cells, loss of function and/or any other symptoms known to be associated with the inflammatory condition. The term will thus be understood to include, inter alia, acute, chronic, ulcerative, specific, allergic and necrotic inflammation, as well as all other forms of inflammation known to those skilled in the art. The term also includes arthritis (including osteoarthritis and rheumatoid arthritis), inflammatory bowel disease, eczema, psoriasis, atopic dermatitis, psoriatic arthropathy and asthma, post operative inflammation, dental inflammation, acute and chronic ocular inflammatory diseases, conjunctivitis. The compounds and compositions of the subject invention advantageously can block the immunogenic inflammatory pathway, thereby providing a method for inhibiting immunogenic inflammation. Accordingly, the subject compounds and compositions can be useful in the treatment of neurogenic inflammation, present in different processes, such as diabetes, asthma, cystitis, gingivitis, migraine, dermatitis, rhinitis, psoriasis, inflammation of sciatic and lumbar nerves, gastrointestinal processes, ocular inflammation, and acute allergic response, asthma, rheumatoid arthritis, osteoarthritis and other inflammatory conditions involving acute and/or chronic joint inflammation in a subject, preferably mammalian, more preferably human.

As used herein, the term “gastrointestinal toxicity” (or “GI toxicity”) includes, but is not limited to, the gastrointestinal side effects associated with the administration of one of more NSAIDs to a subject, such as, for example, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as inflammatory bowel disease, Crohn's disease, gastritis, irritable bowel syndrome, ulcerative colitis, constipation, peptic ulcer, partial enteritis, ulcerative colitis, diverticulitis, gastrointestinal haemorrhagia, regional enteritis, diverticulitis, gastrointestinal lesions, gastrointestinal bleeding, coagulation disorders, hypoprothrombinemia, hemophilia or other bleeding problems, kidney disease and the like. Furthermore, the term “gastrointestinal toxicity” refers to any disease, condition, or disorder, or a symptom of a disease, condition, or disorder related to a decrease in one or more prostaglandins (e.g., PGE₂ and PGI₂) anywhere in the gastrointestinal tract. In a particular embodiment, the term “gastrointestinal toxicity” refers to any disease, condition, or disorder, or a symptom of a disease, condition, or disorder related to a decrease in one or more prostaglandins (e.g., PGE₂ and PGI₂) in the stomach.

The compositions of the present invention have “pain alleviating properties,” “inflammation alleviating properties,” as well as “GI toxicity alleviating properties.” The term “pain alleviating properties” is generally defined herein to include the expressions “pain-suppressing,” “pain-reducing,” and “pain-inhibiting” as the invention is applicable to the alleviation of existing pain, as well as the suppression or inhibition of pain which would otherwise ensue from an imminent pain-causing event. Similarly, the term “inflammation alleviating properties” is generally defined herein to include the expressions “inflammation-suppressing,” “inflammation-reducing,” and “inflammation-inhibiting” as the invention is applicable to the alleviation of existing inflammation, as well as the suppression or inhibition of inflammation which would otherwise ensue from an imminent inflammation -causing event. Furthermore, the term “GI toxicity alleviating properties” is generally defined herein to include the expressions “GI toxicity-suppressing,” “GI toxicity-reducing,” and “GI toxicity-inhibiting” as the invention is applicable to the alleviation of existing GI toxicity, as well as the suppression or inhibition of GI toxicity which would otherwise ensue from an imminent GI toxicity-causing event (e.g., the administration of an NSAID to a subject).

The term “treatment” or “treating,” as used herein, is defined as the application or administration of a therapeutic agent, e.g., a pharmaceutical composition of the invention, to a subject, or application or administration of a therapeutic agent to an isolated tissue or cell line from a subject (e.g., for diagnosis or ex vivo applications), who suffers from pain and/or inflammation, a symptom of pain and/or inflammation or a predisposition toward pain and/or inflammation, with the purpose to cure, heal, alleviate, relieve, alter, remedy, ameliorate, improve or affect the pain and/or inflammation, the symptoms of the pain and/or inflammation or the pain and/or inflammation itself. Furthermore, the term refers to administration of a therapeutic agent, e.g., a pharmaceutical composition of the invention, to a subject, or application or administration of a therapeutic agent to an isolated tissue or cell line from a subject (e.g., for diagnosis or ex vivo applications), who suffers from GI toxicity, a symptom of GI toxicity or a predisposition toward GI toxicity, with the purpose to cure, counteract, heal, alleviate, relieve, alter, remedy, ameliorate, improve or affect the GI toxicity, the symptoms of the GI toxicity or the GI toxicity itself. In particular embodiments, the GI toxicity to be treated is associated with administration of one or more NSAIDs. Such treatments may be specifically tailored or modified, based on knowledge obtained from the field of pharmacogenomics.

The term “subject” includes living organisms in which pain and/or inflammation can occur, or which are susceptible to pain, inflammation and/or GI toxicity. The term “subject” includes animals (e.g., mammals, e.g., cats, dogs, horses, pigs, cows, goats, sheep, rodents, e.g., mice or rats, rabbits, squirrels, bears, primates (e.g., chimpanzees, monkeys, gorillas, and humans)), as well as chickens, ducks, geese, and transgenic species thereof, and cells derived therefrom. In a preferred embodiment, the term “subject” refers to a human. In one embodiment, the subject is a human with an increased risk for GI toxicity, such as one suffering from diabetes.

Administration of the compositions of the present invention to a subject to be treated can be carried out using known procedures, at dosages and for periods of time effective to treat pain and/or inflammation, or GI toxicity, in the subject. In certain embodiments, the GI toxicity in the subject is associated with NSAID use. An effective amount of the therapeutic compositions necessary to achieve a therapeutic effect may vary according to factors such as the state of the disease or disorder in the subject, the age, sex, and weight of the subject, and the ability of the therapeutic compositions to inhibit the pain and/or inflammation in the subject. Dosage regimens can be adjusted to provide the optimum therapeutic response. For example, several divided doses may be administered daily or the dose may be proportionally reduced as indicated by the exigencies of the therapeutic situation. A non-limiting example of an effective dose range for a therapeutic composition or compound of the invention (e.g., an N-methylnicotinamide salt in combination with an NSAID) is between 0.25 and 500 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

In particular, the selected dosage level will depend upon a variety of factors including the activity of the particular compound of the present invention employed, the time of administration, the rate of excretion of the particular NSAID being employed, the duration of the treatment, other drugs, compounds or materials used in combination with the particular composition employed, the age, sex, weight, condition, general health and prior medical history of the patient being treated, and like factors well known in the medical arts.

A medical doctor, e.g., physician or veterinarian, having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The regimen of administration can affect what constitutes an effective amount. The therapeutic formulations can be administered to the subject either prior to or after the onset of pain and/or inflammation or GI toxicity. For example, an N-methylnicotinamide salt can be administered with an NSAID to counter the GI toxicity associated with NSAID use, or N-methylnicotinamide can be administered alone, for example, after administration of an NSAID. Further, several divided dosages, as well as staggered dosages, can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

In particular embodiments, it is especially advantageous to formulate compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a pain, inflammation and/or GI toxicity in subjects.

Compounds of the Invention

Nicotimamide, as well as the nicotinamide derivatives of the invention can be synthesized using techniques well-known the one skilled in the art of organic synthesis.

In one aspect, the nicotinamide derivatives of the instant invention are represented by the Formula I: 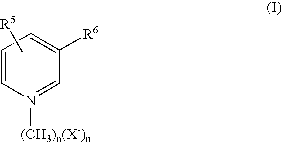
wherein n is 0 or 1; R⁵ is H, F or Cl; R⁶ is azido, —C(O)CHN₂, —C(O)R or the following 5-membered heterocycle: 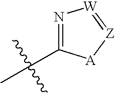
wherein R is NR²R³ or OR⁴; R² and R⁴ each, independently, are hydrogen or C₁₋₄alkyl; R³ is hydrogen, C₁₋₄alkyl or CH₂OH; W and Z are, independently, C(H) or N; A is O, S or NH; and X⁻ is a physiologically suitable counter-anion.

In a particular embodiment of Formula I, n is 1. In yet another embodiment of Formula I, R⁶ is —C(O)R, and R is NR²R³. In still another embodiment of Formula I, R⁶ is —C(O)R, R is NR²R³, and R² represents methyl or hydrogen. In another embodiment of Formula I, R⁶ is —C(O)R, R is NR²R³, and R³ represents CH₂OH or hydrogen. In yet another embodiment of Formula I, R⁶ is —C(O)R, R represents the group OR⁴, and R⁴ represents C14 alkyl. In still another embodiment of Formula I, R⁶ is —C(O)R, R is OR⁴, and R⁴ represents propyl or ethyl.

In a preferred embodiment, the compound of Formula I is selected from a 1-methylnicotinamide salt or a 1-methyl-N′-hydroxymethylnicotinamide salt. In another preferred embodiment, the compound of Formula I is selected from a 1-methylnicotinic acid ethyl ester salt or a 1-methylnicotinic acid propyl ester salt. In another preferred embodiment, the compound of Formula I is selected from a 1-methylnicotinic acid salt. In another embodiment of Formula I, n is 1, and X⁻ is chloride, benzoate, salicylate, acetate, citrate or lactate.

In yet another preferred embodiment, the compound of Formula I is selected from 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride or 1-methylnicotinic acid propyl ester chloride.

In another embodiment of Formula I, R⁶ is the 5-membered heterocycle, wherein A is O, and W and Z are CH; A is S, and W and Z are CH; A is NH, and W and Z are CH; A is O, W is N and Z is CH; A is S, W is N and Z is CH; A is NH, W is N and Z is CH; A is O, W is CH and Z is N; A is S, W is CH and Z is N; A is NH, W is CH and Z is N; A is O, Wand Z are CH; A is S, W and Z are CH; or A is NH and W and Z are N. In a preferred embodiment of Formula I wherein R⁶ is a 5-membered heterocycle, n is 1.

In another embodiment of Formula I, n is 1, R⁵ is H and R⁶ is azido.

The present invention also relates to prodrugs of nicotimamide and the nicotinamide derivatives of Formula I disclosed herein, as well as pharmaceutical compositions comprising such prodrugs. For example, compounds of the invention which include acid or hydroxyl functional groups can also be prepared and administered as a corresponding ester with a suitable alcohol or acid. The ester can then be cleaved by endogenous enzymes within the subject to produce the active agent.

In a preferred embodiment of the invention, the nicotinamide derivatives of Formula I, alone or in combination with one or more NSAIDs, exhibit pain and/or inflammation alleviating properties in subject. In another preferred embodiment of the invention, the nicotinamide derivatives of Formula I, alone or in combination with one or more NSAIDs, counteract GI toxicity associated with administration of NSAIDs. In a preferred embodiment, the subject is a mammal. In another preferred embodiment, the subject is a human.

In a particular embodiment, nicotimamide, N-methylnicotinamide and its derivatives are used as cytoprotective agents (i.e., “gut-sparing”).

A particularly preferred pharmaceutical composition of the invention consists of an N-methylnicotinamide salt and naprosyn for use in the treatment of pain and/or inflammation in a subject in need thereof.

Another particularly preferred pharmaceutical composition of the invention consists of an N-methylnicotinamide salt and voltaren for use in the treatment of pain and/or inflammation in a subject in need thereof.

Another particularly preferred pharmaceutical composition of the invention consists of an N-methylnicotinamide salt and indomethacin for use in the treatment of pain and/or inflammation in a subject in need thereof.

Another particularly preferred pharmaceutical composition of the invention consists of an N-methylnicotinamide salt and diclofenac for use in the treatment of pain and/or inflammation in a subject in need thereof.

In another embodiment, the compounds of formula I are selective prostaglandin mimetics.

In yet another embodiment, the compounds of formula I can be used to treat gastrointestinal toxicity in a subject.

Another particularly preferred pharmaceutical composition of the invention consists of an N-methylnicotinamide salt and aspirin for use in the treatment of pain and/or inflammation in a subject in need thereof.

Another particularly preferred pharmaceutical composition of the invention consists of an N-methylnicotinamide salt and aspirin for use in the treatment of gastrointestinal toxicity in a subject in need thereof.

In another embodiment, a compound of Formula I can be administered to a subject to counteract the adverse side-effects, e.g., gastrointestinal toxicity, of an NSAID. In yet another embodiment, the chloride salt of NMA is administered to a subject to counteract the adverse side-effects, e.g., gastrointestinal toxicity, of an NSAID. In still another embodiment NMA is administered to a subject to treat gastrointestinal toxicity

As used herein, the language “pharmaceutically acceptable salt” refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Preparing a “pharmaceutically acceptable salt” results in the compound of Formula (I) having a “physiologically suitable counter-anion,” e.g., Cl.

The term “alkyl” refers to saturated aliphatic groups, including straight-chain alkyl groups, branched-chain alkyl groups, cycloalkyl, heterocyclyl, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably has 20 or fewer carbon atoms in the backbone. Likewise, preferred cycloalkyls have from 3-10 carbon atoms in their ring structure, and more preferably have, 3-8 carbon atoms in their ring structure and even more preferably have 5, 6 or 7 carbons in the ring structure. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl.

Moreover, alkyl (e.g., methyl, ethyl, propyl, butyl, pentyl, hexyl, etc.) include both “unsubstituted alkyl” and “substituted alkyl,” the latter of which refers to alkyl moieties having substituents replacing a hydrogen on one or more carbons of the hydrocarbon backbone, which allow the molecule to perform its intended function.

Examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃₀(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃H (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R′)₀₋₃R′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. In certain embodiments, a carbonyl moiety (C═O) may be further derivatized with an oxime moiety, e.g., an aldehyde moiety may be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

Combination Therapies

The prostaglandin mimetics (e.g., a PGI₂ agonist or a PGE₂ agonist or nicotinamide derivative, e.g., MNA) of the present invention are intended to be useful, e.g., in the methods of present invention, alone or in combination with one or more additional compounds useful for treating pain and/or inflammation-related diseases and disorders (e.g., NSAIDs). The prostaglandin mimetics (e.g., a PGI₂ agonist or a PGE₂ agonist or nicotinamide derivative, e.g., MNA) of the present invention are intended to be useful for counteracting the deleterious side effects associated with NSAID administration. These additional compounds may comprise compounds of the present invention or compounds, e.g., commercially available compounds, known to treat, prevent, or reduce the symptoms of pain and/or inflammation-related diseases and disorders.

Additionally, the nicotinamide derivatives of the present invention are intended to be useful, e.g., in the methods of present invention, alone or in combination with one or more additional compounds useful for treating GI toxicity. The invention also provides the combination of a prostaglandin mimetic (e.g., a PGI₂ agonist or a PGE₂ agonist or nicotinamide derivative, e.g., MNA) with one or more additional compounds useful for treating GI toxicity. These additional compounds may comprise compounds of the present invention or compounds, e.g., commercially available compounds, known to treat, prevent, or reduce the symptoms of pain and/or inflammation-related diseases and disorders.

In particular, the nicotinamide derivatives of Formula I can be co-administered with an NSAID. Examples of NSAIDs useful for the instant invention include, but are not limited to, aceclofenac, acemetacin, acetaminophen, acetaminosalol, acetyl-salicylic acid, acetyl-salicylic-2-amino-4-picoline-acid, 5-aminoacetylsalicylic acid, alclofenac, aminoprofen, amfenac, ampyrone, ampiroxicam, anileridine, bendazac, benoxaprofen, bermoprofen, α-bisabolol, bromfenac, 5-bromosalicylic acid acetate, bromosaligenin, bucloxic acid, butibufen, carprofen, celecoxib, chromoglycate, cinmetacin, clindanac, clopirac, sodium diclofenac, diflunisal, ditazol, droxicam, enfenamic acid, etodolac, etofenamate, felbinac, fenbufen, fenclozic acid, fendosal, fenoprofen, fentiazac, fepradinol, flufenac, flufenamic acid, flunixin, flunoxaprofen, flurbiprofen, glutametacin, glycol salicylate, ibufenac, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, montelukast, mycophenolic acid, nabumetone, naproxen, niflumic acid, nimesulide, olsalazine, oxaceprol, oxaprozin, oxyphenbutazone, paracetamol, parsalmide, perisoxal, phenyl-acethyl-salicylate, phenylbutazone, phenylsalicylate, pyrazolac, piroxicam, pirprofen, pranoprofen, protizinic acid, reserveratol, salacetamide, salicylamide, salicylamide-O-acetyl acid, salicylsulphuric acid, salicin, salicylamide, salsalate, sulindac, suprofen, suxibutazone, tamoxifen, tenoxicam, theophylline, tiaprofenic acid, tiaramide, ticlopridine, tinoridine, tolfenamic acid, tolmetin, tropesin, xenbucin, ximoprofen, zaltoprofen, zomepirac, tomoxiprol, zafirlukast and cyclosporine. Most preferred are indomethacin, voltaren and naprosyn.

The following references, hereby incorporated by reference herein, describe various NSAIDs suitable for use in the invention described herein, and processes for their manufacture: U.S. Pat. No. 3,558,690 to Sallmann and Pfister, (assigned to Ciba Geigy), issued 1971; U.S. Pat. No. 3,843,681 (assigned to American Home Products), issued 1974; U.S. Pat. No. 3,766,263 to Godfrey, (assigned to Reckitt and Colman) issued 1973; U.S. Pat. No. 3,845,215 to Godfrey (assigned to Reckitt and Colman) issued 1974; U.S. Pat. No. 3,600,437 to Marshall (assigned to Eli Lilly), issued 1971; U.S. Pat. No. 3,228,831 to Nicholson and Adams, (assigned to Boots Pure Drug), issued 1966; (U.S. Pat. No. 3,385,886 to Nicholson and Adams, (assigned to Boots Pure Drug) issued 1968; U.S. Pat. No. 3,161,654 to Shen, (assigned to Merck & Co.), issued 1964; U.S. Pat. No. 3,904,682 to Fried and Harrison, (assigned to Syntex), issued 1975; U.S. Pat. No. 4,009,197 to Fried and Harrison, (assigned to Syntex), issued 1977; U.S. Pat. No. 3,591,584 to Lombardino (assigned to Pfizer) issued 1971; U.S. Pat. No. 5,068,458 to Dales et al., (assigned to Beecham Group, PLC.), issued Nov. 26, 1991; U.S. Pat. No. 5,008,283 to Blackburn et al. (assigned to Pfizer, Inc.), issued Apr. 16, 1991; and U.S. Pat. No. 5,006,547 to Loose (assigned to Pfizer), issued Apr. 9, 1991.

Preferred NSAIDs are those agents which have been marketed to the public.

In accordance with an aspect of the present invention there is provided a method of achieving a therapeutic effect for treating a patient suffering from pain and/or and inflammation-related disease or disorder comprising administering a therapeutically effective amount of a pharmaceutical combination comprising as active ingredients (i) a prostaglandin mimetic (e.g., a nicotinamide of Formula I, or an acceptable salt thereof); and (ii) an NSAID or a pharmaceutically acceptable salt thereof to the patient.

In accordance with another aspect of the present invention there is provided a method of achieving a therapeutic effect for treating a patient suffering from GI toxicity, as well as an NSAID-associated disease or disorder by administering a therapeutically effective amount of a pharmaceutical combination comprising as active ingredients (i) a prostaglandin mimetic (e.g., a nicotinamide of Formula I, or an acceptable salt thereof); and (ii) an NSAID or a pharmaceutically acceptable salt thereof to the patient. In accordance with another aspect of the present invention there is provided a method of achieving a therapeutic effect for treating a patient suffering from GI toxicity comprising administering a therapeutically effective amount of a pharmaceutical combination comprising as active ingredients (i) a prostaglandin agonist (e.g., a PGI₂ agonist or a PGE₂ agonist), or an acceptable salt thereof; and (ii) an NSAID or a pharmaceutically acceptable salt thereof to the patient.

In another embodiment of this aspect of the present invention the therapeutic effect achieved may be synergistic, in that, the therapeutic effect is greater than the sum of the therapeutic effect achieved by the administration of the active ingredients separately.

In some embodiments, a prostaglandin mimetic (e.g., a nicotinamide of Formula I, or an acceptable salt thereof) and one or more NSAIDs are included in a single composition, which is administered to a subject experiencing pain and/or inflammation, or GI toxicity. In other embodiments, a nicotinamide derivative of Formula I and one or more NSAIDs are administered separately to such a subject. In yet other embodiments, prostaglandin agonist (e.g., a PGI₂ agonist or a PGE₂ agonist) and one or more NSAIDs are included in a single composition, which is administered to a subject experiencing pain and/or inflammation, or GI toxicity. In other embodiments, a prostaglandin agonist (e.g., a PGI₂ agonist or a PGE₂ agonist) and one or more NSAIDs are administered separately to such a subject. The first and at least one second compound may either be co-administered to a subject (i.e., at the same time) or be administered sequentially (i.e., one after the other).

A combination of compounds described herein can either result in synergistic increase in effectiveness against pain and/or inflammation, relative to effectiveness following administration of each compound when used alone, or such an increase can be additive. Compositions described herein typically include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to each compound when used alone.

A synergistic effect can be calculated, for example, using suitable methods such as, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

Each of the components of the pharmaceutical composition of the invention ((i) a prostaglandin mimetic (e.g., a nicotinamide of Formula I, or an acceptable salt thereof); and (ii) an NSAID) for administration can individually be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

In some embodiments, dose of a nicotinamide derivative of the invention for treatment of pain and/or inflammation or GI toxicity is between about 0.0001 mg and about 25 mg. In some embodiments, a dose of a nicotinamide derivative of the invention used in compositions described herein is less than about 100 mg, or less than about 80 mg, or less than about 60 mg, or less than about 50 mg, or less than about 30 mg, or less than about 20 mg, or less than about 10 mg, or less than about 5 mg, or less than about 2 mg, or less than about 0.5 mg. Similarly, in some embodiments, a dose of a second compound (i.e., an NSAID) as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg.

In one embodiment, the prostaglandin mimetic (e.g., a compound of Formula I, e.g., MNA) is administered first followed by administration of an NSAID. In another embodiment, the NSAID is administered first followed by administration of a prostaglandin mimetic (e.g., a compound of Formula I, e.g., MNA). In still another embodiment, the prostaglandin mimetic (e.g., a compound of Formula I, e.g., MNA) and NSAID are administered at the same time.

Formulations for Administration

In another embodiment, the present invention is directed to a packaged pharmaceutical composition comprising a container holding a therapeutically effective amount of a prostaglandin mimetic (e.g., a nicotinamide of Formula I, or an acceptable salt thereof), alone and in combination with one or more NSAIDs; and instructions for using the compound to treat, prevent, or reduce one or more symptoms of one or more pain, inflammation and/or GI toxicity related conditions in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing one or more pain, inflammation and/or GI toxicity-related abnormalities in a subject.

Another embodiment of the invention is a pharmaceutical composition comprising a therapeutically effective amount of a nicotinamide compound of Formula I and a pharmaceutically acceptable carrier.

The language “therapeutically effective amount” describes the amount of nicotinamide derivative of Formula I of the invention that is effective to treat one or more symptoms of pain and/or inflammation in a subject.

The language “pharmaceutically acceptable carrier” includes a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. In one embodiment, the pharmaceutically acceptable carrier is not DMSO alone.

The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For example, for oral administration the compounds can be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ film coating systems available from Colorcon, West Point, Pa. (e.g., OPADRY™ OY Type, OY-C Type, Organic Enteric OY-P Type, Aqueous Enteric OY-A Type, OY-PM Type and OPADRY™ White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

Transmucosal Administration

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

Transurethal Administration

With regard to transurethal administration, the formulation can comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C 10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

Transrectal Administration

Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration may be in the range of from about 10 minutes to about 6 hours, e.g., less than about 3 hours.

Vaginal or Perivaginal Administration

Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, 20th Edition, A. Gennaro (ed.) (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration may be in the range of from about 10 minutes to about 6 hours, e.g., less than about 3 hours.

Intranasal or Inhalation Administration

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 μm, e.g., from about 1 μm to about 25 μm.

Topical Formulations

Topical formulations can be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

Intrathecal Administration

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

Intravesical Administration

The term intravesical administration is used herein in its conventional sense to mean delivery of a drug directly into the bladder. Suitable methods for intravesical administration can be found, for example, in U.S. Pat. Nos. 6,207,180 and 6,039,967.

Additional Administration Forms

Additional dosage forms of this invention include dosage forms as described in U.S. Pat. No. 6,340,475, U.S. Pat. No. 6,488,962, U.S. Pat. No. 6,451,808, U.S. Pat. No. 5,972,389, U.S. Pat. No. 5,582,837, and U.S. Pat. No. 5,007,790. Additional dosage forms of this invention also include dosage forms as described in U.S. patent application Ser. No. 20030147952, U.S. patent application Ser. No. 20030104062, U.S. patent application Ser. No. 20030104053, U.S. patent application Ser. No. 20030044466, U.S. patent application Ser. No. 20030039688, and U.S. patent application Ser. No. 20020051820. Additional dosage forms of this invention also include dosage forms as described in PCT Patent Application WO 03/35041, PCT Patent Application WO 03/35040, PCT Patent Application WO 03/35029, PCT Patent Application WO 03/35177, PCT Patent Application WO 03/35039, PCT Patent Application WO 02/96404, PCT Patent Application WO 02/32416, PCT Patent Application WO 01/97783, PCT Patent Application WO 01/56544, PCT Patent Application WO 01/32217, PCT Patent Application WO 98/55107, PCT Patent Application WO 98/11879, PCT Patent Application WO 97/47285, PCT Patent Application WO 93/18755, and PCT Patent Application WO 90/11757.

Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles for example, by injection or in the form of wafers or discs by implantation.

In a preferred embodiment of the invention, the nicotinamide compounds of Formula I, are administered to a subject, alone or in combination with an NSAID, using a sustained release formulation.

The term delayed release is used herein in its conventional sense to refer to a drug formulation that provides for an initial release of the drug after some delay following drug administration which, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

Dosing

The therapeutically effective amount or dose of a compound of the present invention will depend on the age, sex and weight of the patient, the current medical condition of the patient and the nature of the pain, inflammation and/or GI-toxicity being treated. The skilled artisan will be able to determine appropriate dosages depending on these and other factors. A suitable dose of a compound of the present invention can be in the range of from about 0.001 mg to about 1000 mg per day, such as 0.001 mg to about 500 mg per day, such as from about 0.01 mg to about 100 mg, for example, from about 0.05 mg to about 50 mg, such as about 0.5 mg to about 25 mg per day. The dose can be administered in a single dosage or in multiple dosages, for example from 1 to 4 or more times per day. When multiple dosages are used, the amount of each dosage can be the same or different. For example a dose of 1 mg per day can be administered as two 0.5 mg doses, with about a 12 hour interval between doses.

It is understood that the amount of compound dosed per day can be administered every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Exemplification of the Invention

The invention is further illustrated by the following example, which could be used to examine prostaglandin-releasing activity of the compounds of the invention. The example should not be construed as further limiting. The animal models used throughout the Examples are accepted animal models and the demonstration of efficacy in these animal models is predictive of efficacy in humans.

### EXAMPLE 1

Thrombolytic activity of the prostaglandin mimetics of the invention can be assessed using the assay described in WO 2005/067927. This assay was used to demonstrate that 1-methyl-3-acetylpyridinium salt, 1-methylnicotinamide chloride and 1-methyl-N′-(hydroxymethyl)nicotinamide have the ability to induce the release of prostacyclin in animal models.

### EXAMPLE 2

In this study, streptozocin (70 mg/kg injected intraperitoneally) was used to induce diabetes mellitus and non-diabetic and diabetic rats were pretreated 30 min prior to exposure to aspirin (ASA; 150 mg/kg in 0.1 N HCl i.g.) or 3.5 h of water immersion and restraint stress (WRS) with 1) vehicle (saline) or 2) MNA (2.5-50 mg/kg i.g.). The area and number of gastric lesions was determined by planimetry, gastric blood flow (GBF) was examined by H₂-gas clearance technique, and mieloperoxidase (MPO) activity, activity of superoxide dismutase (SOD) and the malonyldialdehyde (MDA) concentration as an index of lipid peroxidation were determined in gastric mucosa using ELISA. The gene expression IL-1β and TNF-α in the gastric mucosa and their plasma levels were evaluated by RT-PCR and ELISA, respectively. ASA and WRS caused typical gastric lesions and reduced significantly GBF (by 35% and 28% from basal) and increased MPO activity (2-3 fold) and gastric mucosal MDA content and these effects were significantly augmented in diabetic rats (plasma glucose≦400 mg/dL). The ulcerogenic effects of ASA or WRS were accompanied by the fall in GBF and the overexpression of mRNAs for TNF-α and IL-1, as well as a marked increase in their plasma levels. MNA dose-dependently attenuated ASA- and WRS-induced gastric erosions in diabetic and non-diabetic animals while raising GBF in intact and injured gastric mucosa by about 22% and 33%, respectively, and caused downregulation of IL-1β and TNF-α. The mucosal SOD activity was significantly decreased while the MDA activity was significantly increased in ASA- and WRS-exposed animals without or with diabetes and these effects were markedly attenuated by MNA. These studies demonstrate that 1) diabetes enhances the susceptibility of gastric mucosa to the damage induced by ASA and WRS via mechanism involving fall in GBF, suppression of SOD activity, an increase in lipid peroxidation and overexpression of proinflammatory cytokines IL-1β and TNF-α, and 2) MNA affords protection against ASA- and WRS-induced damage. This experiment is also described in an abstract published for Digestive Disease Week 2006 (May 20-25, 2006, Los Angeles, Calif.; http://www.medscape.com/viewprogram/5452).

Equivalents

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

Incorporation by Reference

The contents of all references, issued patents, and published patent applications cited throughout this application are hereby expressly incorporated by reference in their entireties. It should be understood that the use of any of the compounds described herein are within the scope of the present invention and are intended to be encompassed by the present invention and are expressly incorporated herein for all purposes.

1. A pharmaceutical composition comprising a Non-Steroidal Anti-Inflammatory Drug (NSAID) and a prostaglandin mimetic.

2. The pharmaceutical composition of claim 1, wherein the prostaglandin mimetic is selected from the group consisting of genistein, daidzein, tamoxifen, tetrandrine, thapsigargin, and a compound of formula (I): 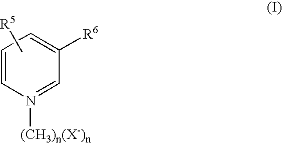
wherein
n is 0 or 1;
R⁵ is H, F or Cl;
R⁶ is azido, —C(O)CHN₂, —N(H)C(O)NH₂, —N(H)C(O)H, —C(O)R or the following 5-membered heterocycle: 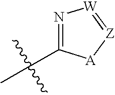
wherein
R is NR²R³ or OR⁴;
R² and R⁴ each, independently, are hydrogen or C₁₋₄alkyl;
R³ is hydrogen, C₁₋₄alkyl or CH₂OH;
W and Z are, independently, C(H) or N;
A is O, S or NH; and
X⁻ is a physiologically suitable counter-anion.

3. The pharmaceutical composition of claim 1, wherein the prostaglandin mimetic is a prostacyclin mimetic.

4. The pharmaceutical composition of claim 1, wherein the prostaglandin mimetic is a prostaglandin agonist.

5. The composition of claim 4, wherein the prostaglandin agonist is selected from the group consisting of a PGI₂ agonist and a PGE₂ agonist.

6. The composition of claim 4, wherein the agonist is selected from the group consisting of U46619, I-BOP, STA₂, BW245C, L-644698, ZK110841, 13,15-dihydro-15-keto-PGD₂, indomethacin, 15-R-methyl-PGD₂, 15d-PGJ₂, ONO-KI-004, iloprost, 17-phenyl-trinor PGE₂, butaprost, 11-deoxy PGE1, AH13205, ONO-AEI-259, sulprostone, MB28767, misoprostol, SC46275, ONO-AE-249, PGE₁-OH, ONO-AEI-329, cicaprost, carbacyclin, fluprostenol, latanoprost, travoprost, bimatoprost, beraprost, cloprostenol sodium, eicosopentanoic acid, docosohexanoic acid, ceramide, sodium butyrate and aluminum fluoride.

7. A method of treating or preventing the deleterious effects associated with NSAID administration in a subject said method comprising administering of a pharmaceutical composition comprising a Non-Steroidal Anti-Inflammatory Drug (NSAID) and a prostaglandin mimetic.

8. The method of claim 7, wherein the deleterious effects associated with NSAID administration in a subject are related to a decrease in one or more prostaglandins anywhere in the gastrointestinal tract.

9. The method of claim 8, wherein the deleterious effects associated with NSAID administration in a subject are related to a decrease in one or more prostaglandins in the stomach.

10. The method of claim 8, wherein the prostaglandins are PGE₂ and/or PGI₂.

11. The method of claim 7, wherein the deleterious effects associated with NSAID administration in a subject are caused by gastro-intestinal (GI) toxicity.

12. The method of claim 11, wherein the GI toxicity is selected from the group consisting of gastritis, peptic erosions, ulceration, gastric lesions and GI bleeding.

13. The pharmaceutical composition of claim 1, wherein the NSAID is selected from the group consisting of aspirin, indomethacin, voltaren, naprosyn, ibuprofen, naproxen, fenoprofen, tolmetin, sulindac, meclofenamate, ketoprofen, piroxicam, flurbiprofen, diclofenac, acetylsalicylic acid, sodium acetylsalicylic acid, calcium acetylsalicylic acid, salicylic acid, sodium salicylate, choline salicylate, magnesium salicylate, salsalate, sodium salicylate, diflunisal, ketorolac, carprofen, mefenamic acid, meloxicam and nimesulide.

14. A method of treating pain and/or inflammation in a subject, said method comprising administering to the subject in need thereof a therapeutically-effective amount of a pharmaceutical composition comprising an NSAID and a molecule that selectively stimulates the release of a prostaglandin.

15. The method of claim 14, wherein the molecule is selected from the group consisting of genistein, daidzein, tamoxifen, tetrandrine, thapsigargin and a compound of formula (I): 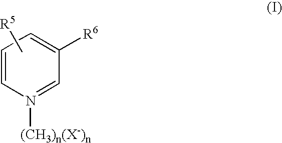
wherein
n is 0 or 1;
R⁵ is H, F or Cl;
R⁶ is azido, —C(O)CHN₂, —N(H)C(O)NH₂, —N(H)C(O)H, —C(O)R or the following 5-membered heterocycle: 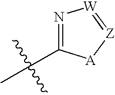
wherein
R is NR²R³ or OR⁴;
R² and R⁴ each, independently, are hydrogen or C₁₋₄alkyl;
R³ is hydrogen, C₁₋₄alkyl or CH₂OH;
W and Z are, independently, C(H) or N;
A is O, S or NH; and
X⁻ is a physiologically suitable counter-anion.

16. The method of claim 14, wherein the prostaglandin is prostacyclin.

17. A method of treating pain and/or inflammation in a subject, said method comprising administering to the subject in need thereof a therapeutically-effective amount of a pharmaceutical composition comprising an NSAID and a prostaglandin agonist.

18. The method of claim 17, wherein the prostaglandin agonist is selected from the group consisting of a PGI₂ agonist and a PGE₂ agonist.

19. The method of claim 17, wherein the agonist is selected from the group consisting of U46619, I-BOP, STA₂, BW245C, L-644698, ZK110841, 13,15-dihydro-15-keto-PGD₂, indomethaein, 15-R-methyl-PGD₂, 15d-PGJ₂, ONO-KI-004, iloprost, 17-phenyl-trinor PGE₂, sulprostone, butaprost, I 1-deoxy PGE₁, AH13205, ONO-AEI-259, MB28767, misoprostol, SC46275, ONO-AE-249, PGE₁-OH, ONO-AEI-329, cicaprost, carbacyclin, fluprostenol, latanoprost, travoprost, bimatoprost, beraprost, cloprostenol sodium, eicosopentanoic acid, docosohexanoic acid, ceramide, sodium butyrate and aluminum fluoride.

20. The method of claim 14, wherein the NSAID is selected from the group consisting of aspirin, indomethacin, voltaren, naprosyn, ibuprofen, naproxen, voltaren, fenoprofen, tolmetin, sulindac, meclofenamate, ketoprofen, piroxicam, flurbiprofen, diclofenac, acetylsalicylic acid, sodium acetylsalicylic acid, calcium acetylsalicylic acid, salicylic acid, sodium salicylate, choline salicylate, magnesium salicylate, salsalate, sodium salicylate, diflunisal, ketorolac, carprofen, mefenamic acid, meloxicam and nimesulide.

21. The method of claim 14, wherein the inflammation is selected from the group consisting of fever, arthritis, asthma, bronchitis, menstrual cramps, tendinitis, bursitis, inflammatory disorders of the skin, gastrointestinal conditions, vascular diseases, migraine headaches, periarteritis nodosa, thyroiditis, aplastic anemia, Hodgkin's disease, sclerodoma, rheumatic fever, myasthenia gravis, sarcoidosis, nephrotic syndrome, Behcet's syndrome, polymyositis, hypersensitivity, conjunctivitis, gingivitis, swelling occurring after injury and myocardial ischemia.

22. The method of claim 21, wherein the arthritis is selected from the group consisting of rheumatoid arthritis, spondyloarthopathies, gouty arthritis, systemic lupus erythematosus, osteoarthritis and juvenile arthritis.

23. The method of claim 21, wherein the inflammatory disorders of the skin are selected from the group consisting of psoriasis, eczema, burns and dermatitis.

24. The method of claim 21, wherein the gastrointestinal conditions are selected from the group consisting of inflammatory bowel syndrome, Crohn's disease, gastritis, irritable bowel syndrome and ulcerative colitis.

25. The method of claim 14 wherein the pain is selected from the group consisting of menstrual pain, low back pain, neck pain, skeletal pain, post-partum pain, headache, pain associated with migraine, toothache, sprains, strains, arthritis, degenerative joint diseases, gout, ankylosing spondylitis, bursitis, burns, including radiation and corrosive chemical injuries, sunburns, bone fracture, immune and autoimmune diseases, cellular neoplastic transformations or metastic tumor growth, and pain following surgical and dental procedures.

26. (canceled)

27. The method of claim 15, wherein the molecule is a compound of formula (I) and wherein said pain and/or inflammation is related to gastric lesion, symptoms of gastro-intestinal (GI) toxicity, ischemia, reperfusion, colorectal cancer, or damage to the gastric mucosa in the subject.

28. The method of claim 27, wherein the symptoms of GI toxicity are selected from the group consisting of gastric lesion, gastritis, peptic erosion, ulceration, and GI bleeding.

29. The pharmaceutical composition of claim 2, wherein the prostaglandin mimetic is a compound of formula (I) and n is 1.

30. The pharmaceutical composition of claim 2, wherein the prostaglandin mimetic is a compound of formula (I) and R⁶ is —C(O)R, and R is NR²R³.

31. The pharmaceutical composition of claim 2, wherein the prostaglandin mimetic is a compound of formula (I) and R⁶ is —C(O)R, R is NR²R³, and R² represents methyl or hydrogen.

32. The pharmaceutical composition of claim 2, wherein the prostaglandin mimetic is a compound of formula (I) and R⁶ is —C(O)R, R is NR²R³, and R³ represents CH₂OH or hydrogen.

33. The pharmaceutical composition of claim 2, wherein the prostaglandin mimetic is a compound of formula (I) and R⁶ is —C(O)R, R represents the group OR⁴, and R⁴ represents C₁₋₄ alkyl.

34. The pharmaceutical composition of claim 2, wherein the prostaglandin mimetic is a compound of formula (I) and R⁶ is —C(O)R, R represents the group OR⁴, and R⁴ represents propyl or ethyl.

35. The pharmaceutical composition of claim 2, wherein the prostaglandin mimetic is a compound of formula (I) and is selected from the group consisting of a 1-methylnicotinamide salt and a 1-methyl-N′-hydroxymethylnicotinamide salt.

36. The pharmaceutical composition of claim 2, wherein the prostaglandin mimetic is a compound of formula (I) and is selected from the group consisting of a 1-methylnicotinic acid ethyl ester salt and a 1-methylnicotinic acid propyl ester salt.

37. The pharmaceutical composition of claim 2, wherein the prostaglandin mimetic is a compound of formula (I) and is a 1-methylnicotinic acid salt.

38. The pharmaceutical composition of claim 2, wherein the prostaglandin mimetic is a compound of formula (I), n is 1, and X⁻ is chloride, benzoate, salicylate, acetate, citrate or lactate.

39. The pharmaceutical composition of claim 2, wherein the prostaglandin mimetic is a compound of formula (I) and is selected from the group consisting of 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride and 1-methylnicotinic acid propyl ester chloride.

40. The pharmaceutical composition of claim 2, wherein the prostaglandin mimetic is a compound of formula (I) and R⁶ is a 5-membered heterocycle selected from the group wherein A is O, and W and Z are CH; A is S, and W and Z are CH; A is NH, and W and Z are CH; A is O, W is N and Z is CH; A is S, W is N and Z is CH; A is NH, W is N and Z is CH; A is O, W is CH and Z is N; A is S, W is CH and Z is N; A is NH, W is CH and Z is N; A is O, W and Z are CH; A is S, W and Z are CH; and A is NH and W and Z are N.

41. The pharmaceutical composition of claim 40, wherein n is 1.

42. The pharmaceutical composition of claim 2, wherein the prostaglandin mimetic is a compound of formula (I), n is 1, R⁵ is H and R⁶ is azido.

43. The method of claim 27 wherein the compound of formula (I) is 1-methylnicotinamide.

44. The method of claim 43, wherein the symptoms of GI toxicity are selected from the group consisting of gastric lesion, gastritis, peptic erosion, and ulceration, and GI bleeding.

45. A method of treating an inflammation-associated disorder and/or pain in a subject in need thereof by administering to the subject a pharmaceutical composition comprising an NSAID and 1-methylnicotinamide, and pharmaceutically acceptable salts thereof.

46. The method of claim 14, wherein the NSAID is selected from the group consisting of aspirin, indomethacin, voltaren, naprosyn, ibuprofen, naproxen, voltaren, fenoprofen, tolmetin, sulindac, meclofenamate, ketoprofen, piroxicam, flurbiprofen, diclofenac, acetylsalicylic acid, sodium acetylsalicylic acid, calcium acetylsalicylic acid, salicylic acid, sodium salicylate, choline salicylate, magnesium salicylate, salsalate, sodium salicylate, diflunisal, ketorolac, carprofen, mefenamic acid, meloxicam and nimesulide.

47. The method of claim 15 wherein the molecule is a compound of formula (I) and n is 1.

48. The method of claim 15 wherein the molecule is a compound of formula (I) and R⁶ is —C(O)R, and R is NR²R³.

49. The method of claim 15 wherein the molecule is a compound of formula (I) and R⁶ is —C(O)R, R is NR²R³, and R² represents methyl or hydrogen.

50. The method of claim 15 wherein the molecule is a compound of formula (I) and R⁶ is —C(O)R, R is NR²R³, and R³ represents CH₂OH or hydrogen.

51. The method of claim 15 wherein the molecule is a compound of formula (I) and R⁶ is —C(O)R, R represents the group OR⁴, and R⁴ represents C₁₋₄ alkyl.

52. The method of claim 15 wherein the molecule is a compound of formula (I) and is selected from the group consisting of a 1-methylnicotinamide salt and a 1-methyl-N′-hydroxymethylnicotinamide salt.

53. The method of claim 15 wherein the molecule is a compound of formula (I) and is selected from the group consisting of a 1-methylnicotinic acid ethyl ester salt and a 1-methylnicotinic acid propyl ester salt.

54. The method of claim 15 wherein the molecule is a compound of formula (I) and is selected from the group consisting of 1-methylnicotinic acid salts.

55. The method of claim 15 wherein the molecule is a compound of formula (I), n is 1, and X⁻ is chloride, benzoate, salicylate, acetate, citrate or lactate.

56. The method of claim 15 wherein the molecule is a compound of formula (I) and is selected from the group consisting of 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride or 1-methylnicotinic acid propyl ester chloride.

57. The method of claim 15 wherein the molecule is a compound of formula (I) and R⁶ is a 5-membered heterocycle selected from the group wherein A is O, and W and Z are CH; A is S, and W and Z are CH; A is NH, and W and Z are CH; A is O, W is N and Z is CH; A is S, W is N and Z is CH; A is NH, W is N and Z is CH; A is O, W is CH and Z is N; A is S, W is CH and Z is N; A is NH, W is CH and Z is N; A is O, W and Z are CH; A is S, W and Z are CH; and A is NH and W and Z are N.

58. The method of claim 58, wherein n is 1.

59. The method of claim 15 wherein the molecule is a compound of formula (I), n is 1, R⁵ is Hand R⁶ is azido.
